(19) United States
(12) Patent Application Publication
(10) Pub. No.: US 2021/0346649 A1
(43) Pub. Date: Nov. 11, 2021

(54) MAGNETIZABLE CLAMP FOR A CATHETER

(71) Applicant: Weinberg Medical Physics Inc, North Bethesda, MD (US)

(72) Inventor: Irving WEINBERG, North Bethesda, MD (US)

(21) Appl. No.: 17/317,354

(22) Filed: May 11, 2021

Related U.S. Application Data
(60) Provisional application No. 63/022,886, filed on May 11, 2020.

(51) Int. Cl. A61M 25/01; A61B 34/30

(52) U.S. Cl. CPC A61M 25/0158; A61B 2034/301; A61B 34/30; A61M 25/0127

(57) ABSTRACT

Disclosed embodiments provide an apparatus and method for converting a catheter to a magnetic catheter and using the magnetic catheter by coupling a clamp with magnetizable components to a catheter.

### CROSS REFERENCE AND PRIORITY CLAIM

This application claims priority to U.S. Provisional Patent Application Ser. No. 63/022,886, entitled “MAGNETIZABLE CLAMP FOR A CATHETER” filed May 11, 2020, the entirety of which is incorporated by reference.

### FIELD

Disclosed embodiments are directed, generally, to a method and apparatus for making and using a magnetizable clamp for a catheter.

### BACKGROUND

Many catheters exist for medical interventions. These catheters generally do not have magnetic properties. It is known that a catheter can be manipulated in a body if the catheter has a magnetic tip and the magnetic tip is exposed to a magnetic field applied from a location that is external to the body, for example U.S. Pat. No. 6,428,551 “Magnetically navigable and/or controllable device for removing material from body lumens and cavities”.

### SUMMARY

Disclosed embodiments provide an apparatus and method for converting a catheter to a magnetic catheter and using the magnetic catheter by coupling a clamp with magnetizable components to a catheter.

In accordance with at least one embodiment, the clamp includes interlocking components, and locking components for securing the clamp to the catheter. The magnetizable components are coupled to or integrally formed with the interlocking components.

In accordance with at least one embodiment an external magnetic field is applied to a clamped portion of a catheter is inside a body, and the external magnetic field manipulates the clamped portion of the catheter via the magnetizable components.

### BRIEF DESCRIPTION OF FIGURES

FIG. 1 illustrates a top view of a clamp apparatus in the open position including interlocking components, locking components, and components made of magnetizable material;

FIG. 1A illustrates a cross-sectional view of the clamp apparatus in an open position according to the disclosed embodiments;

FIG. 1B illustrates a cross-sectional view of the clamp apparatus in the closed position on a catheter according to the disclosed embodiments;

FIG. 2 depicts use of the clamp apparatus clamped on a catheter with an external magnetic field generator according to the disclosed embodiments; and

FIG. 3 is a flowchart of a method of use of the clamp according to the disclosed embodiments.

### DETAILED DESCRIPTION

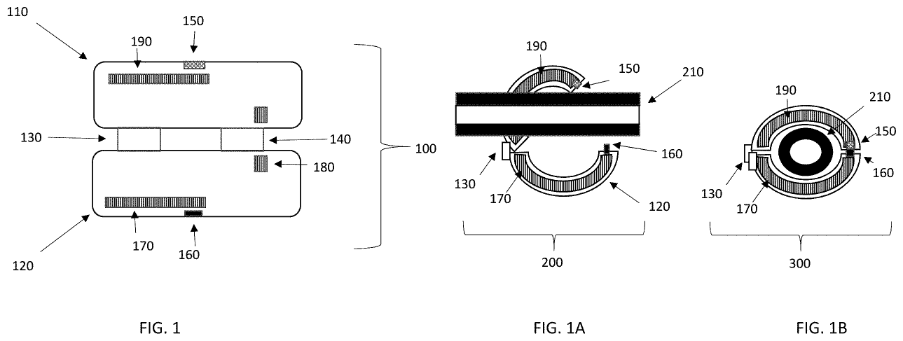

FIG. 1 shows a side view of closed clamp apparatus 100, including interlocking components 110 and 120, shown with hinges 130 and 140, and locking components 150 and 160 that can close clamp 100 either permanently or temporarily (for example through application of a key). The locking mechanism may be fitted or frictional or some other method. Components 170, 180, 190 made of magnetizable material (for example AlNiCo) are attached to or form part of interlocking components 110 and 120. The illustrated arrangement and number of magnetizable components in FIG. 1 is not meant to be limiting, and other arrangements and numbers of the magnetizable components may be used. For example, part 170 may extend to the entire length of the clamp, or may be shorter than that length. Clamp apparatus 200 is shown in a cross-sectional view in FIG. 1A, surrounding non-magnetic catheter 210 but still in an open position. Clamp apparatus 300 is shown in a cross-sectional view in a closed position around catheter 210 in FIG. 1B. As shown in FIGS. 1A and 1B, the apparatus consists of a clamp 100 surrounding a non-magnetic catheter 210. The apparatus 100, 200, 300 is used to convert non-magnetic catheter 210 into a magnetic catheter that can be manipulated by applying a magnetic field to the components 170, 180, 190 that are part of the clamp apparatus 100.

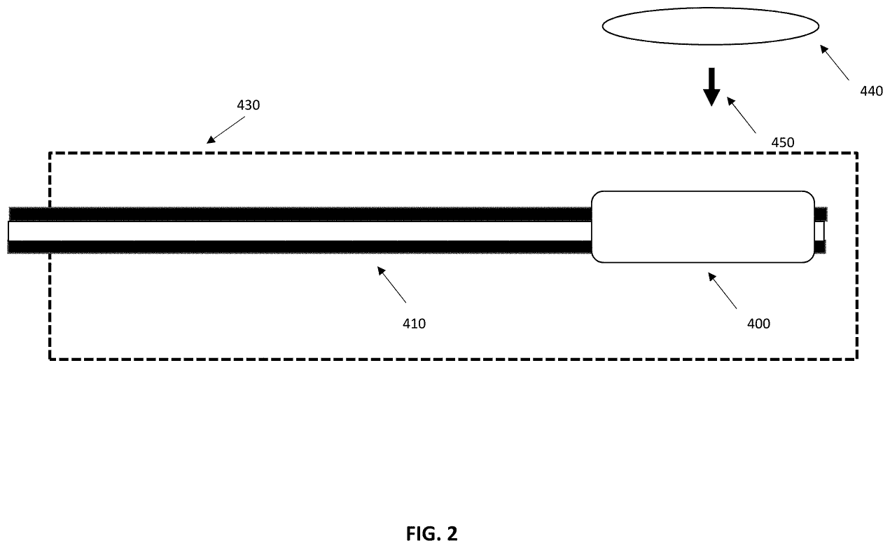

FIG. 2 illustrates the use of the clamp apparatus 400 clamped on a catheter 410 that may be inserted into in a body of a living being (for example an animal or human) 430. Outside the body 430 is a magnetic field generator (for example a coil or electropermanent magnet) 440, which created a magnetic field 450 that penetrates the body 430 and exerts a force and/or torque on the apparatus 400, thereby manipulating the catheter tip.

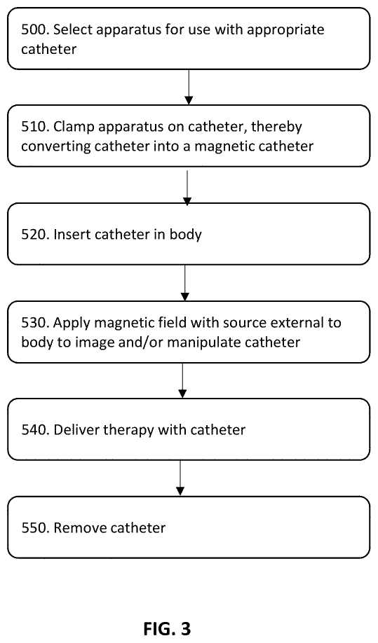

FIG. 3 provides a method for using a clamp apparatus, starting with step 500 in which a user selects an appropriate clamp apparatus for a catheter. Then, the user may affix the clamp apparatus onto catheter in step 510. Catheter and clamped apparatus are inserted into body in step 520. A magnetic field is applied to at least one of the magnetizable components in the clamp in order to image and/or manipulate catheter in step 530. Therapy or diagnostic procedure is delivered via catheter, for example with a drug or removing tissue for biopsy or destroying cancer cells or other useful tasks in step 540. Then catheter is removed from body in step 550.

It is understood that the arrangement of magnetic components 150 and 160 as shown in the figure is not limited to that shown, and that many other configurations of magnetizable materials are possible within the purview of the invention. The magnetizable materials may be in shapes that can be controlled independently with different forms and/or components of externally applied magnetic fields, as described by U.S. patent invention application Ser. No. 14/930,126, entitled “Method and Apparatus for Non-Contact Axial Particle Rotation and Decoupled Particle Propulsion” incorporated by reference.

The magnetizable components may be pre-polarized so that they may be pushed by an externally applied magnetic field, as described by U.S. Pat. No. 9,380,959, entitled “MRI-guided Nanoparticle Cancer Therapy Apparatus and Methodology” and related patents incorporated by reference.

It is understood that the magnetic components 170, 180, 190 may be different for different versions of the apparatus, so that an external magnetic field applied to various catheters or sections of catheters with the clamps applied may have different mechanical responses.

It is understood that an appropriately changing magnetic field (for example 100 kHz alternating magnetic field) may heat the magnetic components if desired by the practitioner.

It is understood that the body may be within the field-of-view of an imaging instrument (for example a magnetic resonance imaging device) and the clamp may therefore be visible in the images, thereby assisting in localization of the catheter by a practitioner viewing the image, or by a computer guiding therapy. The catheter may be manipulated by a magnetic system that also provides imaging of the catheter. The magnetic system may be robotic, meaning that the operation may be either autonomous or semi-autonomous.

It is understood that the magnetic polarization and magnitude of the magnetic materials in the apparatus may be nulled by externally applying a rapidly changing magnetic field, for example in order to keep catheters from sticking to one another or to staples in the body.

It is understood that the apparatus is rendered sterile (for example by irradiation) and may be applied to clamp on a catheter by a medical or veterinary practitioner in a clinic (“in the field”). Alternatively, the apparatus may be applied to clamp on a catheter in a factory and shipped with the catheter to the practitioner's site.

It is understood that the term “catheter” is not limited to a tube for carrying fluids, but encompasses more generally a flexible rod or wire that may be hollow or not hollow, may be electrically conductive or non-conductive, and may have tools attached to it, for example a cutting tool or heating section. The catheter may have a cuff for wedging against an airway, as may be useful for bronchoalveolar lavage procedures. The term “lavage” is meant to include the administration and subsequent removal of fluid from tissues or organs containing tissues (for example, the lung).

The catheter may admit a guidewire or one or more optical fibers or an antenna or may be introduced into the body through another tube. The catheter may be used to remove tissue (for example by suction) to biopsy a region of the body or to remove unwanted tissue (for example a cancer). The catheter may itself serve as a guidewire for another catheter, in which case either of those catheters may receive a clamp.

It is understood that the hinges 130 and 140 and locking components 150 and 160 shown are for illustration, and that other means of locking the apparatus in place onto a catheter are included in the invention.

It is understood that the term “clamp” means that the apparatus encloses or partially encloses a catheter and fixes the apparatus in place along the length of the catheter. It is understood that more than one clamp apparatus may be affixed to a catheter.

It is understood that the clamped potion of the catheter is to be inserted into a body.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

1. An apparatus comprising:
a clamp having magnetizable components which clamps to a catheter,
wherein the clamped portion of the catheter is configured to be inserted into a body, and
wherein an application of a magnetic field to a body manipulates the catheter by acting on at least one of the magnetizable components within the clamp.

2. The apparatus of claim 1, wherein the clamp further comprises hinges.

3. The apparatus of claim 1, wherein the clamp further comprises interlocking components and the magnetizable components are integrally formed with the interlocking components or affixed to the interlocking components.

4. The apparatus of claim 3, wherein the clamp further comprises locking components configured to releasably or permanently lock the interlocking components together around the catheter.

5. The apparatus of claim 1, wherein at least one of magnetizable components is configured to be controlled independently by the application of the magnetic field.

6. The apparatus of claim 1, wherein the magnetizable components are pre-polarized.

7. A method of converting a catheter into a magnetic catheter and using the magnetic catheter comprising:
affixing a clamp containing magnetizable components to a catheter for use when an external magnetic field is applied to a body.

8. The method of claim 7 further comprising delivering therapy to tissues in a body via the magnetic catheter.

9. The method of claim 8 wherein the therapy is lavage.

10. The method of claim 7 wherein tissue is removed from a body via the magnetic catheter.

11. The method of claim 7, further comprising using an external magnetic field to image the magnetic catheter in the body.

12. The method of claim 7, where the affixing of the clamp is performed in the field.

13. The method of claim 7, where manipulation of the catheter is done by a system that also provides images of the catheter.

14. The method of claim 13, where the manipulation is performed robotically.

15. The method of claim 7, wherein the clamp is affixed via locking components configured to releasably or permanently lock interlocking components together around the catheter.
